(19) United States
(12) Patent Application Publication
(10) Pub. No.: US 2026/0283773 A1
(43) Pub. Date: Sep. 24, 2026

(54) IMPLANTABLE PROSTHESIS

(71) Applicant: Davol Inc., Warwick, RI (US)

(72) Inventors: Ian K. Parker, Bristol, RI (US); Greg Daniel Learn, West Warwick, RI (US)

(73) Assignee: Davol Inc., Warwick, RI (US)

(21) Appl. No.: 19/086,453

(22) Filed: Mar. 21, 2025

(51) Int. Cl. A61F 2/00; A61F 5/445

(52) U.S. Cl. CPC A61F 2/0063; A61F 5/445; A61F 2002/0081; A61F 2005/4455; A61F 2230/0058

(57) ABSTRACT

Embodiments disclosed herein relate to an implantable prosthesis for preventing, reinforcing, and/or mending an anatomical defect, including a parastomal hernia which may occur following formation of a stoma or ostomy opening. The implantable prosthesis may include a body having a preformed channel and an opening that is offset from an end of the channel. The channel may be configured to receive a portion of a bowel through the offset opening such that the bowel portion is lateralized and stabilized in the channel.

### FIELD

Disclosed embodiments are related to an implantable prosthesis, and more particularly to a prothesis for mending defects and weaknesses of soft tissue and muscle walls.

### BACKGROUND

A defect in a muscle or tissue wall, such as a hernia, is commonly repaired with an implantable prosthesis that is configured to cover and/or fill the defect. For some procedures, an implantable repair fabric, such as a mesh fabric, is sutured, stapled, tacked, or otherwise provisionally anchored in place over, under, or within the defect. Tissue integration with the fabric, such as tissue ingrowth into and/or along the mesh fabric, eventually completes the repair. Various repair fabrics are known and used for repairing soft tissue and muscle wall defects. Such fabrics are oftentimes fabricated from monofilaments that are knitted into a mesh having pores or interstices that promote tissue ingrowth and integration with the fabric.

### SUMMARY

In some embodiments, an implantable prosthesis is provided and comprises a prosthesis body and a channel formed in the prosthesis body. The channel may include a first end portion and a second end portion, and the channel may have a three-dimensional configuration. In some embodiments, an opening may be formed in the channel at a location spaced apart from the first end portion, and the opening may be configured to receive a portion of a lateralized bowel such that the portion of the lateralized bowel passes through the opening and is positioned in the channel.

In some embodiments, a method of repairing and/or preventing a parastomal hernia is provided. The method may comprise placing an implantable prosthesis adjacent to a stoma. In some embodiments, the implantable prosthesis may comprise a prosthesis body and a channel formed on the prosthesis body having a three-dimensional configuration. The method may further comprise passing a portion of a bowel through an opening formed in the channel such that the portion of the bowel is lateralized and received in the channel. In some embodiments, the opening may be formed at a location spaced apart from an end portion of the channel.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A is a bottom perspective view of an implantable prosthesis, according to some embodiments;

FIG. 1B is a top perspective view of the implantable prosthesis of FIG. 1A, according to some embodiments;

FIG. 1C is a bottom view of the implantable prosthesis of FIG. 1A, according to some embodiments;

FIG. 1D is a side view of the implantable prosthesis of FIG. 1A, according to some embodiments;

FIG. 1E is a schematic illustration of an offset opening formed in a channel of the implantable prosthesis of FIGS. 1A-1D, according to some embodiments;

FIG. 2A is a schematic cross-sectional illustration of a parastomal hernia repair with the implantable prosthesis of FIGS. 1A-1E receiving a lateralized bowel; and

FIG. 2B is a schematic illustration of a parastomal hernia repair employing the implantable prosthesis of FIGS. 1A-1E.

### DETAILED DESCRIPTION

The present disclosure is directed to an implantable prosthesis for preventing, reinforcing and/or mending an anatomical defect, and is particularly suitable for preventing, reinforcing and/or mending defects in, and weaknesses of, soft tissue and muscle walls or other anatomical regions. The phrase “mending a defect” includes acts of repairing, augmenting, and/or reconstructing a defect and/or a potential defect. For ease of understanding, and without limiting the scope of the invention, the embodiments of a prosthesis described herein are particularly employed in connection with a parastomal hernia which may occur following the formation of a stoma or ostomy opening, such as formed in conjunction with an ileostomy or colostomy. For example, the embodiments disclosed herein may be employed for use in retro rectus or pre-peritoneal parastomal hernia repair. A parastomal hernia is a type of incisional hernia that occurs at or adjacent to a stoma. It should be understood, however, that the embodiments of a prosthesis disclosed herein are not so limited and may be employed in other anatomical procedures, as should be apparent to one of skill in the art. For example, and without limitation, the prosthesis may be employed for use in ostomy reinforcement (i.e., parastomal hernia prevention), or other suitable repair procedures.

Such defects (e.g., parastomal hernias) may be repaired using various techniques, including the Sugarbaker technique. This repair involves a surgeon performing a laparotomy. Following hernia reduction, sac resection and reduction of the stoma trephine, the ostomy opening is covered with an intraperitoneally placed prosthetic mesh that is sutured to the fascia. The bowel is lateralized and secured between the mesh and the peritoneum, thereby lateralizing the forces which press the bowel ventrally onto the abdominal wall, shifting them from pushing up toward the defect and causing these forces to press ventrally against an intact abdominal wall.

The inventors have recognized that during the course of repairing a parastomal hernia, it is beneficial to support a portion of bowel that is located adjacent to the stoma. In addition, the inventors have recognized that a traditional Sugarbaker technique may not fully secure the bowel such that a portion of the bowel may be allowed to shift while received in the channel. In some instances, shifting of the bowel in the channel may cause abrasion of the bowel against the walls of the channel and/or the defect against which the prosthesis is positioned. For example, such abrasion may occur near the open end of the channel when a lateralized bowel is received in a channel of a traditional Sugarbaker prosthesis.

To address the problems with traditional Sugarbaker designs, the inventors have found that it is desirable to provide a prosthesis capable of reducing forces applied to a lateralized bowel as well as shifting of the bowel in the channel. It is also desirable to provide a prosthesis that allows for a gradual transition of the lateralized bowel into the channel to avoid excess stresses on the bowel itself. In view of the above, the inventors have appreciated benefits associated with an implantable prosthesis having a channel and an opening that is offset from an end portion of the channel. In such configurations, the inventors have appreciated that by passing the bowel portion through the offset opening, that the bowel may experience additional lateral and medial reinforcement while positioned in the channel. In addition, the inventors have found that the offset opening may secure an orientation of the bowel once received in the channel such that shifting of the bowel is reduced or eliminated.

In some embodiments, an implantable prosthesis according to the embodiments disclosed herein includes a prosthesis body and a channel preformed with the body. In some such embodiments, the implantable prosthesis may have a preformed, three dimensional configuration. The implantable prosthesis may also include an opening spaced from an end portion of the channel. The offset opening may be configured to receive at least a portion of a bowel that is lateralized within the channel such the opening is configured to retain a position and orientation of the bowel portion. In some embodiments, the offset opening may be constructed and arranged as a cruciate opening. That is, the offset opening may have a central opening and one or more slits or channels extending outwardly therefrom. Optionally, one or more periphery openings may be positioned at the end of the one or more slits or channels. The inventors have appreciated that a cruciate opening may permit insertion and retention of a bowel portion through the opening once the bowel portion is lateralized in the channel. In addition, the inventors have appreciated that a cruciate opening may allow for the opening to accommodate bowel portions of different sizes. That is, the presence of slits or channels in the opening may allow the prosthesis material surrounding the opening to splay outwardly and adjust to the size of the bowel portion. In some embodiments, the opening may not be constructed and arranged as a cruciate opening as other suitable shapes are contemplated, as disclosed herein.

The inventors have appreciated that the embodiments of an implantable prosthesis disclosed herein may provide a variety of benefits. These benefits include that the offset opening may be configured to increase support of a patient's bowel while also limiting or preventing damage to the bowel caused by adhesion, erosion, and/or excess stress concentration due to the lateralization of the bowel in the channel. Thus, the use of an implantable prosthesis according to embodiments disclosed herein may improve the efficacy of various repair procedures (e.g., parastomal hernia repair) of anatomical defects in patients.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

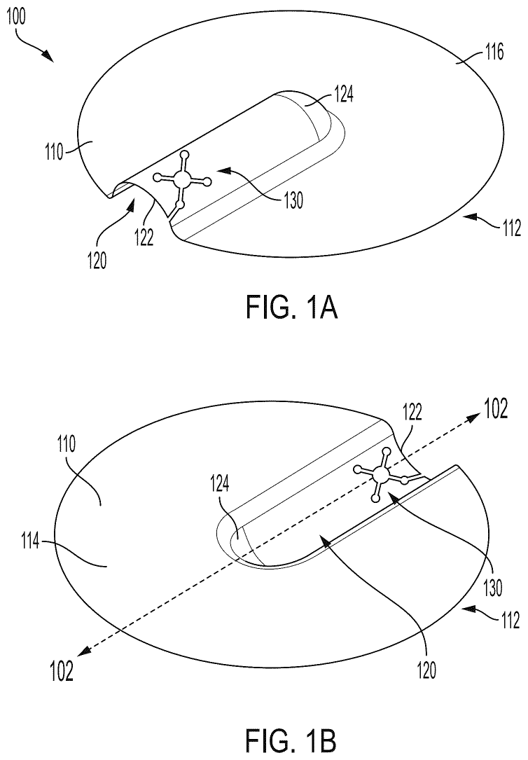

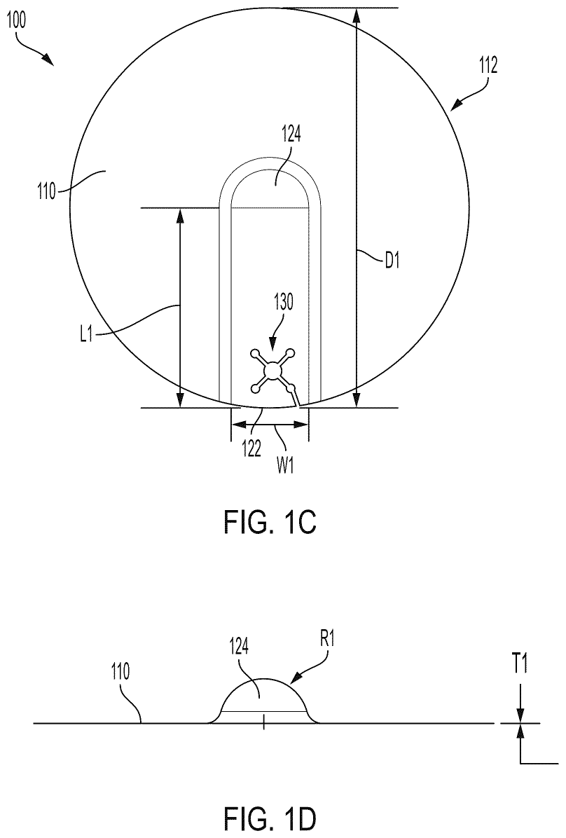

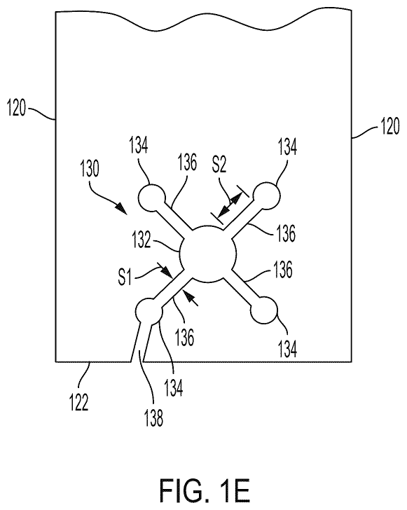

FIGS. 1A-1E illustrate various views of an embodiment of an implantable prosthesis 100 for covering, reinforcing, and/or mending tissue and muscle wall defects including, but not limited to, a defect in the peritoneum. The implantable prosthesis 100 may include a prosthesis body 110 and a channel 120 preformed with the body. In this manner, the implantable prosthesis may have a preformed, three dimensional configuration. The channel 120 may be formed such that it is recessed relative to a planar top surface 114 of the prosthesis and protrudes past a planar bottom surface 116 of the prosthesis.

In some embodiments, the prosthesis body 110 may include a repair fabric which is relatively flexible, thin and light weight and meets the performance and physical characteristics for covering, reinforcing and/or mending soft tissue and muscle wall defects. The prosthesis body 110 may be configured with a size and/or shape suitable to cover or extend across the opening or weakness in tissue and/or muscle when the body is placed against the tissue and/or muscle wall with the defect.

In some embodiments, the channel 120 may be configured to receive a portion of a lateralized bowel during a parastomal hernia repair procedure, where the channel may be formed using the Sugarbaker technique. In some embodiments, the prosthesis may include an adhesion resistant barrier located on the inner side of the channel to isolate the portion of the lateralized bowel from channel surface. In this manner, the adhesion resistant barrier may reduce, if not eliminate, potential adhesions between the prosthesis body and the lateralized bowel. In other embodiments, however, the channel may not include an adhesion resistant coating.

In some embodiments, the channel 120 may be arranged to extend from the outer periphery 112 to an inner region of the body. The channel may include an first open end portion 122 at the outer periphery and a second closed end portion 124 opposite the open end portion. The closed end portion 124 of the channel may be configured and arranged to overlie the ostomy opening through which the bowel portion may be passed. As illustrated in FIGS. 1A-1D, the channel 120 may have a partial tubular configuration with a semicircular shape within a plane oriented perpendicular to the length of the channel, where the length of the channel is in the direction of longitudinal axis 102. The closed end portion 124 of the channel may be configured with a partial hemispherical shape and be located proximate a center of the body.

As illustrated in FIGS. 1A-1E, the preformed channel 120 may extend in a radial direction from the outer periphery 112 to the center of the body. As illustrated, the channel may be configured to extend linearly from the open end portion 122 to the closed end portion 124. However, it is to be appreciated that the channel may be preformed to have any suitable configuration as should be apparent to one of skill in the art. For example, the channel may be configured to extend in a non-linear (e.g., curved or tortuous) fashion from the open end portion 122 to the closed end portion 124. The channel may also be of any suitable cross-sectional shape (e.g., a semicircle that extends at a suitable length to form a channel) as the disclosure is not so limited. In some embodiments, the channel may be constructed and arranged to be of a complementary shape to the lateralized bowel.

In some embodiments, the portion of the body 110 excluding the channel 120 may have a planar configuration prior to implantation. The body may have sufficient flexibility to conform to the anatomy adjacent the ostomy upon implantation to repair the hernia defect. In other embodiments, the portion of the body extending outwardly from the channel may have a non-planar configuration, such as a preformed curved configuration, including a concave or convex shape relative to the channel, which may also have a desired amount of flexibility.

The preformed channel may be formed using a variety of molding techniques. For example, the preformed channel may be formed in the body using a cold forming technique. In this manner, a sheet of body material may be compressed in a mold, such as a clamshell mold, to plastically deform the sheet and form the channel in a desired shape and size defined by the mold. In some embodiments, the channel may be formed using a heat molding technique where the channel may be compressed in a mold and then heated to an appropriate temperature for example, between the glass transition temperature and melting temperature, to set the configuration of the channel. Therefore, the resulting device may include a channel that may maintain a desired shape and overall configuration in an unloaded state. If desired, the body may also be preformed in a similar manner to have a non-planar configuration. It is to be appreciated that other fabrication processes may be employed to preform the channel and/or non-planar body as should be apparent to one of skill in the art.

The prosthesis body 110 and the channel 120 may be configured to have any shape and/or size suitable for a particular application and/or to accommodate a lateralized bowel of various sizes as should be understood by one of skill in the art.

In the embodiments illustrated in FIGS. 1A-1E, the prosthesis body 110 may be configured with a circular shape. However, it is to be appreciated that the prosthesis may employ a body having any suitable shape for a particular application. For example, and without limitation, the body may be configured with a quadrilateral shape, such as a square or rectangle, a triangular shape, a polygonal shape, or an ovoid shape.

In some embodiments, the prosthesis body may have a maximum width D1 of greater than or equal to 10 cm, 15 cm, 20 cm, 25 cm, 30 cm, 35 cm, 40 cm, 45 cm, or greater. Likewise, in some embodiments, the prosthesis body may have a maximum width D1 of lesser than or equal to 50 cm, 45 cm, 40 cm, 35 cm, 30 cm, 25 cm, 20 cm, 15 cm, or lesser. For a body with a circular shape, the maximum width corresponds to the diameter.

In some embodiments, a channel formed in the prosthesis body may have a length L1 of greater than or equal 3 cm, 4 cm, 5 cm, 6 cm, 7 cm, 8 cm, 9 cm, 10 cm, 11 cm, 12 cm, 12.5 cm, 13 cm, 14 cm, or greater. Likewise, in some embodiments, the channel may have a length L1 of lesser than or equal to 15 cm, 14 cm, 13 cm, 12.5 cm, 12 cm, 11 cm, 10 cm, 9 cm, 8 cm, 7 cm, 6 cm, 5 cm, 4 cm, or lesser. The channel formed in the prosthesis body may also have a maximum width W1 of greater than or equal to 1 cm, 1.5 cm, 2 cm, 2.5 cm, 3 cm, 3.5 cm, 4 cm, 4.5 cm, 5 cm, 5.5 cm, 6 cm, 6.5 cm, or greater. Likewise, the channel may have a maximum width W1 of less than or equal to 7 cm, 6.5 cm, 6 cm, 5.5 cm, 5 cm, 4.5 cm, 4 cm, 3.5 cm, 3 cm, 2.5 cm, 2 cm, or lesser.

The channel may have any suitable radius of curvature R1 about its longitudinal axis. In some embodiments, a suitable radius of curvature R1 may be greater than or equal to 1 cm, 1.5 cm, 2 cm, 2.25 cm, 2.5 cm, 2.75 cm, 3 cm, 3.5 cm, 4 cm, 4.5 cm, or greater. In other embodiments, a suitable radius of curvature R1 is lesser than or equal to 5 cm, 4.5 cm, 4 cm, 3.5 cm, 3 cm, 2.75 cm, 2.5 cm, 2.25 cm, 2 cm, 1.5 cm, or lesser.

Although various sizes are disclosed above by way of example, the prosthesis body and the channel formed in the body may be of any suitable size as the disclosure is not so limited. Ranges of the foregoing are also contemplated. For example, the maximum width D1 of the prosthesis body may be between 15 cm and 25 cm, the length L1 may be between 7.5 cm and 12.5 cm, the width W1 may be between 3 cm and 7 cm, and the radius of curvature R1 may be between 1.5 cm and 4.5 cm.

The sizes of the prosthesis body and channel may also vary according to the size of the repair site and the size of the bowel portion to be received within the channel. For example, in some embodiments for a small bowel, the prosthesis body may have a maximum width D1 between 15 cm and 20 cm, and the channel may have a length L1 between 7.5 cm and 10 cm and a radius of curvature R1 of approximately 1.5 cm. In another example for a large bowel or a caecum, the prosthesis body may have a maximum width D1 between 20 cm and 25 cm, and the channel may have a length L1 between 10.5 cm and 12.5 cm and a radius of curvature R1 of approximately 4.5 cm.

The prosthesis body and/or the channel may also have any suitable thickness T1 as the disclosure is not so limited. In some embodiments, a suitable thickness may be greater than or equal to approximately 0.25 mm, 0.5 mm, 0.75 mm, 1 mm, 1.5 mm, 2 mm, 2.5 mm, 3 mm, 4 mm, or greater. Likewise, in some embodiments, a suitable thickness may be lesser than or equal to approximately 5 mm, 4 mm, 3 mm, 2.5 mm, 2 mm, 1.5 mm, 1 mm, 0.75 mm, 0.5 mm, or lesser. In some embodiments, the channel and the planar portion of the prosthesis body may have the same thickness. In other embodiments, however, the channel and the planar portion of the prosthesis body have different thicknesses.

The prosthesis body 110 and/or channel 120 of the prosthesis may be constructed of any suitable material as the disclosure is not so limited. In some embodiments, the prosthesis may be constructed out of a monofilament (e.g., polypropylene or polyester) such that the prosthesis is non-resorbable. Alternatively, the prosthesis may be constructed out of a resorbable material such as a mesh fabric having a knit construction that promotes tissue infiltration through pores of the mesh. The repair fabric may also have sufficient flexibility to promote an easy reduction in size for entry into the subject. In this manner, the flexible fabric may be collapsed into a slender configuration, such as a roll, which can be supported in, and advanced through, a narrow laparoscopic cannula for use in laparoscopic or robotic procedures.

In some embodiments, the channel 120 may be constructed of a different material than the prosthesis body 110. For example, the prosthesis body may be constructed of a resorbable material to promote tissue infiltration while the channel is formed of a non-resorbable material with an optional adhesion resistant layer to limit or prevent adhesion of lateralized bowel to the channel. If the channel is constructed out of a different material than the prosthesis body, the channel may then be secured to the prosthesis body using heat welding, ultrasonic energy, sutures, or any other suitable attachment method.

In some embodiments, the prosthesis 100 may be formed from a layer of which is a resorbable repair fabric having a first surface that supports functional healing via tissue ingrowth and a second surface with a hydrogel barrier that is adhesion resistant to minimize tissue attachment. When implanted, the mesh promotes rapid tissue or muscle ingrowth into and around the mesh structure while minimizing potential attachment to adjacent viscera. The body eventually becomes resorbed at which time the prosthesis is no longer necessary to separate the viscera on one side of the prosthesis from the abdominal wall on the opposite side of the prosthesis. For example, and without limitation, the body may not be resorbed until such time that the parastomal hernia repair has sufficiently healed so as to no longer be susceptible to adhesions with the viscera. It is to be appreciated that any suitable repair fabric providing tissue ingrowth and adhesion resistance may be employed for the body as should be understood by one of skill in the art.

As disclosed herein, the channel may include an adhesion resistant layer. Such an adhesion resistant layer may be formed from a layer of resorbable and hydrophilic adhesion barrier which reduces the incidence of adhesions. It is composed of modified sodium hyaluronate (HA) and carboxymethylcellulose (CMC). The barrier layer eventually becomes resorbed at which time it is no longer necessary to separate the lateralized bowel on one side of the barrier from the repair fabric on the opposite side of the barrier. For example, and without limitation, the barrier layer may not be resorbed until such time that the repair fabric has been resorbed and/or is otherwise no longer susceptible to adhesions with the bowel. It is to be appreciated that any suitable barrier material may be employed for the channel barrier as should be understood by one of skill.

For some applications, the implantable prosthesis 100 may be formed from two or more layers of biocompatible material. For example, and without limitation, the body 110 of the prosthesis may include a first or ingrowth layer of knitted mesh fabric and a second or barrier layer of adhesion resistant material overlying a surface of the mesh fabric. The barrier layer may be separate from and attached to the fabric layer using any suitable fastening technique as should be apparent to one of skill in the art. For example, and without limitation, the mesh fabric and the barrier layer may be joined together by suturing, ultrasonic welding, lamination and/or overmolding.

Examples of resorbable surgical materials which may be utilized for the prosthesis and are suitable for tissue or muscle reinforcement and defect correction include, but are not limited to, PHASIX Mesh (available from Davol, Inc.), polyglactin (VICRYL available from Ethicon, Inc.) and polyglycolic acid (DEXON—available from US Surgical, Inc.). Collagen materials such as COOK SURGISIS, available from Cook Biomedical, Inc. may also be used. Non-resorbable materials, including BARD Mesh (available from Davol, Inc.), BARD Soft Mesh (available from Davol, Inc.), SOFT TISSUE PATCH (microporous ePTFE available from W.L. Gore & Associates, Inc.); SURGIPRO (available from US Surgical, Inc.); TRELEX (available from Meadox Medical); PROLENE and MERSILENE (available from Ethicon, Inc.); and other mesh materials (e.g., available from Atrium Medical Corporation), may be suitable for applications involving permanent correction of tissue or muscle defects. It also is contemplated that the mesh fabric may be formed from multifilament yarns and that any suitable method, such as knitting, weaving, braiding, molding and the like, may be employed to form the mesh material.

For some embodiments employing a separate second layer which may be attached to a first layer, the second layer may be formed from a resorbable layer of SEPRAFILM. A representative and non-limiting sampling of other suitable barrier materials includes a sheet of expanded polytetrafluoroethylene (ePTFE), such as GORETEX available from W.L. Gore & Associates, Inc., having a pore size (submicronal) that discourages tissue ingrowth and adhesion, silicone elastomer, such as SILASTIC Rx Medical Grade Sheeting (Platinum Cured) distributed by Dow Corning Corporation, TEFLON mesh, and microporous polypropylene sheeting (CELGARD). Autogenous, heterogenous and xenogeneic tissue also are contemplated including, for example, pericardium and small intestine submucosa. Absorbable materials, such as oxidized, regenerated cellulose (Intercede (TC7)) may be employed for some applications. It is to be appreciated that any suitable adhesion resistant materials may be used as should be apparent to one of skill in the art.

As shown in FIGS. 1A-1E, the channel 130 may include a first end portion 122 and a second end portion 124. The first end portion 122 may be arranged as an open end portion whereas the second end portion 124 may be arranged as a closed end portion as shown in FIGS. 1B and 1E. The channel 130 may include an offset opening 130 spaced apart from the open end portion 122. That is, the offset opening 130 may be positioned between the open end portion 122 and the closed end portion 124 and formed in a base of the channel. In some embodiments, the offset opening may be positioned closer to the open end portion than the closed end portion to allow for the bowel portion that passes through the opening to become sufficiently lateralized in the channel past the opening before extending through an associated stoma. As disclosed herein, the inventors have appreciated that providing an opening that is offset from an end portion of the channel may provide additional lateral and medial reinforcement of a bowel received in the channel. In addition, the inventors have appreciated that the offset opening may assist in securing a position and orientation of the bowel such that shifting of the bowel is reduced or eliminated once received through the opening in the channel.

Although FIGS. 1A-1E shows the opening 130 as being formed in a base of the channel 130, in some embodiments the opening may be formed in additional prosthesis material that is oriented transverse (e.g., perpendicular) to longitudinal axis 102 of the channel. For example, the opening may be separately formed in a secondary layer of prosthesis material, and then the prosthesis material may be attached to the channel via sutures or other fastening methods such that the secondary layer of prosthesis material extends in a depth direction and width direction across the channel. In addition or alternatively, the implantable prosthesis may include prosthesis material that extends a length of the channel in a direction parallel to the longitudinal axis 102. That is, the channel may include prosthesis material that both extends along a depth and length of the channel to cover the unenclosed portions of the semi-circular channel. In some embodiments, one or more openings may be formed at any suitable location in the portions of the prosthesis material that extend along the length of the channel. In such a configuration, a portion of bowel may be received through the one or more openings to stabilize a position of the bowel and lateralize the bowel in the channel. In addition, although FIGS. 1A-1E show the channel 120 as having an open end portion 122 and a closed end portion 124, in some embodiments the channel may instead be closed at both ends. For example, in configurations where the one or more openings are formed in the prosthesis material that extends along the length of the channel, both end portions of the channel may be closed.

In some embodiments, the opening 130 may be aligned such that the opening is centered about the longitudinal axis 102. In other embodiments, however, the opening 130 may be offset from the longitudinal axis 102. That is, the opening 130 shown in FIGS. 1A-1E may instead be oriented such that the opening 130 is not centered about the longitudinal axis 102 (e.g., the opening 130 shown in FIGS. 1A-1E may be shifted to the left or right of the longitudinal axis 102).

The opening 130 may be offset from the first end portion 122 of the channel 120 at any suitable distance as the disclosure is not so limited. In some embodiments, the opening may be offset at a distance of greater than or equal to 0.5 cm, 1 cm, 1.5 cm, 2 cm, 2.5 cm, 3 cm, 3.5 cm, 4 cm, 4.5 cm, or greater. Likewise, in some embodiments, the opening may be offset at a distance of less than or equal to 5 cm, 4.5 cm, 4 cm, 3.5 cm, 3 cm, 2.5 cm, 2 cm, 1.5 cm, 1 cm, or lesser. In some embodiments, the opening 130 may be offset from the second end portion 124 at a distance of greater than or equal to 2 cm, 2.5 cm, 3 cm, 3.5 cm, 4 cm, 4.5 cm, 5 cm, 5.5 cm, 6 cm, 6.5 cm, 7 cm, 7.5 cm 8 cm, 8.5 cm, 9 cm, or greater. Likewise, in some embodiments, the opening may be offset at a distance of less than or equal to 10 cm, 9.5 cm, 9 cm, 8.5 cm, 8 cm, 7.5 cm, 7 cm, 6.5 cm, 6 cm, 5.5 cm, 5 cm, 4.5 cm, 4 cm, 3.5 cm, 3 cm, or lesser. The distances between the offset opening and the first and second end portions of the channel may be quantified as a ratio. For example, the distance between the offset opening and the first end portion relative to the distance between the offset opening and the second end portion may be a ratio of 1:4, 1:2 3:4, 1:1, 4:3, 2:1, 4:1, or any other suitable ratio as the disclosure is not so limited.

As illustrated in FIGS. 1A-1E, in some embodiments the offset opening 130 may be constructed and arranged as a cruciate opening. The offset opening 130 may include a central hole 132, a plurality of primary slits 136 extending outwardly from central hole 132, and a plurality of corresponding periphery holes 134 located at the end portions of the primary slits 136. The central hole 132 may be the primary hole through which the lateralized bowel is inserted and thus may be larger than the periphery holes. The slits may be arranged to splay outwardly upon insertion of the lateralized bowel such that the central hole and slits collectively form a diamond-like shape that conforms to the size of the bowel portion. The periphery holes 134 may be provided to relieve stress in the opening upon insertion of the bowel by distributing the stress applied to the slits over a larger surface area. While FIGS. 1A-1E show the periphery holes 134, in some embodiments the periphery holes may not be included in the opening 130. In some embodiments, one or more optional secondary slits 138 may be provided that extend outwardly from one or more respective periphery holes 134. These secondary slits can be used to assist in the insertion of the bowel through the offset opening. For example, a surgical instrument may be positioned through the secondary slit 138 to guide the bowel portion through the offset opening. After the bowel portion is received through the opening and into the channel, the secondary slits may be optionally closed either temporarily or permanently (e.g., with sutures). In some embodiments, the secondary slits 138 may extend to the edge of the mesh, e.g., to the open end portion 122, as shown in FIGS. 1A-1E.

Although FIGS. 1A-1E depict a cruciate opening having four primary slits 136 and four periphery holes 134, any suitable number of primary slits and periphery holes may be provided in the opening 130 as the disclosure is not so limited. In some embodiments, the number of slits and/or periphery holes may be greater than or equal to 1, 2, 3, 4, 5, 6, 7, 8, or greater. In some embodiments, the cruciate opening may be oriented such that the opening is formed in an “X-shape” when viewed from the configuration of FIG. 1E. In other embodiments, however, the primary slits 136 and periphery holes 134 are rotated relative to the configuration shown in FIGS. 1A-1E. For example, the opening 130 may be oriented such that the opening is formed in a “plus sign shape” when viewed from the configuration of FIG. 1E. The slits may be of any suitable shape. For example, although FIGS. 1A-1E show linear slits, in some embodiments the primary and/or secondary slits may be formed in a non-linear shape as the disclosure is not so limited.

The opening 130 may be of any suitable shape as the disclosure is not so limited. In some embodiments, the opening 130 may have a cross-shape, a circular shape, a triangular shape, a rectangular shape, a hexagonal shape, or any other suitable shape as the disclosure is not so limited.

The opening 130 may also be of any suitable size as the disclosure is not so limited. In some embodiments, the cruciate-shaped opening shown in FIGS. 1A-1E may be of a size configured to accommodate a given portion of a patient's bowel therethrough. In some embodiments, the opening 130 may have an overall width and/or height dimension (i.e., the dimension spanning from one periphery hole 134 to an opposing periphery hole 134) of greater than or equal to 1.5 cm, 2 cm, 2.5 cm, 3 cm, 3.5 cm, 4 cm, 4.5 cm, 5 cm, 5.5 cm, 6 cm, 6.5 cm, or greater. Likewise, the opening 130 may have an overall width and/or height dimension of less than or equal to 7 cm, 6.5 cm, 6 cm, 5.5 cm, 5 cm, 4.5 cm, 4 cm, 3.5 cm, 3 cm, 2.5 cm, 2 cm, or lesser.

The central hole 132 may have a width dimension of greater than or equal to 0.25 cm, 0.3 cm, 0.35 cm, 0.4 cm, 0.45 cm, 0.5 cm, 0.55 cm, 0.6 cm, 0.65 cm, 0.7 cm, or greater. Likewise, the central hole 132 may have a width dimension of lesser than or equal to 0.75 cm, 0.7 cm, 0.65 cm, 0.6 cm, 0.55 cm, 0.5 cm, 0.45 cm, 0.4 cm, 0.35 cm, 0.3 cm, or lesser. The periphery holes 134 may have a width dimension of greater than or equal to 0.1 cm, 0.15 cm, 0.2 cm, 0.25 cm, 0.3 cm, 0.35 cm, 0.4 cm, 0.45 cm, or greater. Likewise, the periphery holes 134 may have a width dimension of lesser than or equal to 0.5 cm, 0.45 cm, 0.4 cm, 0.35 cm, 0.3 cm, 0.25 cm, 0.2 cm, 0.15 cm, or lesser. For reference purposes herein, the width dimensions for the central hole 132 and the periphery hole 134 may correspond to a diameter of the holes when the shape of the holes is circular. In some embodiments, the periphery holes 134 may be of a smaller size than the central hole 132. When a portion of a bowel is inserted into the central opening 132, the slits 136 and periphery holes 134 may urge the surrounding prosthesis material to splay outwardly to accommodate the size of the bowel portion. The size of the holes and slits may be selected to accommodate different bowel sizes to ensure a snug fit with the bowel when the bowel is inserted through the opening.

Each of the primary slits and/or secondary slits may have a width S1 of greater than or equal to 0.1 mm, 0.15 mm, 0.2 mm, 0.25 mm, 0.3 mm, 0.35 mm, 0.4 mm, 0.45 mm, 0.5 mm, or greater. In some embodiments, each of the primary slits and/or secondary slits may have a length S2 of greater than or equal to 0.3 cm, 0.4 cm, 0.5 cm, 0.6 cm, 0.7 cm, 0.8 cm, 0.9 cm, 1 cm, 1.5 cm, 2 cm, 2.5 cm, or greater. In some embodiments, the opening 130 may be constructed and arranged such that the slits extend along the entirety of the channel width. In other embodiments, as shown in FIG. 1E, the opening may instead be constructed and arranged such that the slits are spaced inwardly from the sides of the channel. The slits may be formed in any suitable fashion, including being laser cut or die cut from a sheet of implantable prosthesis material.

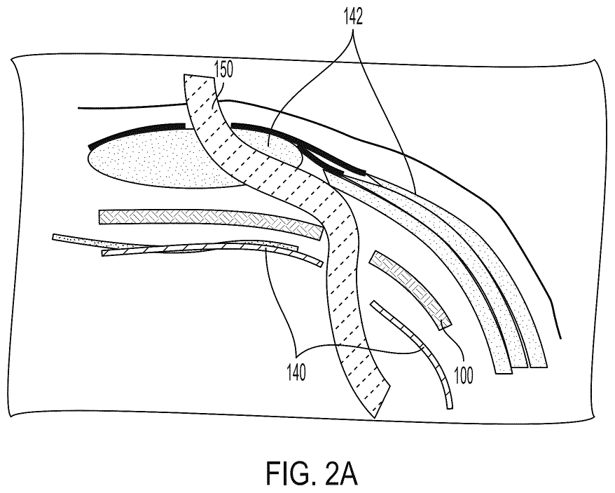

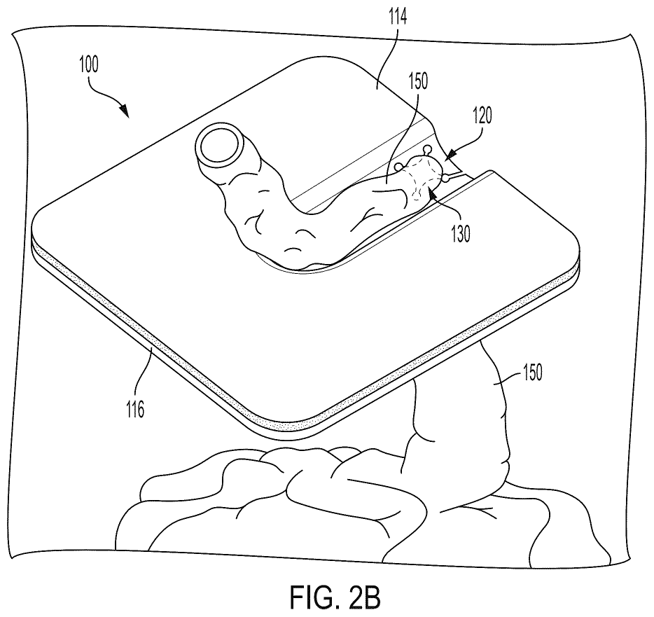

FIGS. 2A-2B illustrate a parastomal hernia repair with the implantable prosthesis of FIGS. 1A-1E positioned over the lateralized bowel.

As indicated above, the prosthesis may be suitable for use during a hernia repair procedure, including a parastomal hernia repair. In one embodiment illustrated in FIGS. 2A-2B, a repair procedure involves the repair of a parastomal hernia using the Sugarbaker technique. As illustrated in FIG. 2A, following hernia reduction, sac resection and/or reduction of the stoma trephine to an appropriate size, if necessary, the prosthesis 100 is secured in the retromuscular plane, e.g., between the peritoneum 140 and the abdominal wall muscle 142 as shown in FIG. 2A. In placing the prosthesis over the ostomy opening in the retromuscular plane, the bowel is lateralized with the preformed channel 120 of the prosthesis receiving a portion of the lateralized bowel 150. In particular, the bowel is lateralized by positioning the bowel 150 through an offset opening 130 formed in the channel 120 (shown in FIGS. 1A-1E and FIG. 2B). The prosthesis may be fastened to the tissue in the retromuscular plane such that the bowel is secured between the prosthesis and the abdominal wall, thereby lateralizing the forces applied to the bowel and causing the applied forces to press against an intact abdominal wall. The preformed shape of the channel may help avoid compression of the bowel which may otherwise occur using a flat prosthesis which is placed over the lateralized bowel and fastened along the sides of the bowel.

As indicated above, the prosthesis may employ a body having any suitable size and/or shape for a particular application. In one embodiment illustrated in FIG. 2A, the body may have a quadrilateral shape, such as a square or rectangle.

In some embodiments, the implantable prosthesis may also have varied topography to assist in securing the prosthesis to the peritoneum. For example, the prosthesis may have micro-surfaces with adhesive or suction properties (e.g., micro suction cups) that secure the prosthesis to the peritoneum. In some embodiments, the varied topography on the prosthesis surface may be provided in conjunction with the surface being tissue infiltratable to both promote tissue ingrowth and increase adhesion.

For purposes of this patent application and any patent issuing thereon, the indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

The use of “including,” “comprising,” “having,” “containing,” “involving,” and/or variations thereof herein, is meant to encompass the items listed thereafter and equivalents thereof as well as additional items.

The embodiments described herein may be embodied as a method. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. An implantable prosthesis comprising:
a prosthesis body;
a channel formed on the prosthesis body, the channel including a first end portion and a second end portion, wherein the channel has a three-dimensional configuration; and
an opening formed in the channel, wherein the opening is formed at a location spaced apart from the first end portion, wherein the opening is configured to receive a portion of a lateralized bowel such that the portion of the lateralized bowel passes through the opening and is positioned in the channel.

2. The implantable prosthesis of claim 1, wherein the first end portion of the channel is configured to be positioned adjacent to a stoma.

3. The implantable prosthesis of claim 1, wherein the opening includes a plurality of slits formed in the channel.

4. The implantable prosthesis of claim 3, wherein the opening is a cruciate opening.

5. The implantable prosthesis of claim 3, wherein the opening includes a first cutout, wherein the plurality of slits extend out from the first cutout, wherein one or more secondary cutouts are formed at an end portion of each slit of the plurality of slits.

6. The implantable prosthesis of claim 1, wherein the channel extends inwardly from an outer periphery of the prosthesis body towards an inner region of the prosthesis body.

7. The implantable prosthesis of claim 6, wherein the prosthesis body includes a planar portion that extends outwardly from a perimeter of the channel towards the outer periphery.

8. The implantable prosthesis of claim 1, wherein the prosthesis body has a width greater than or equal to 10 cm and less than or equal to 50 cm.

9. The implantable prosthesis of claim 1, wherein the opening has a width of greater than or equal to 1.5 cm and less than or equal to 5.5 cm, and a height of greater than or equal to 1.5 cm and less than or equal to 5.5 cm.

10. The implantable prosthesis of claim 1, wherein the channel has a length greater than or equal to 4 cm and less than or equal to 15 cm and a width greater than or equal to 1 cm and less than or equal to 6 cm.

11. The implantable prosthesis of claim 1, wherein the second end portion is a closed end portion.

12. The implantable prosthesis of claim 1, wherein the second end portion is an open end portion.

13. The implantable prosthesis of claim 1, wherein the channel includes an adhesion resistant coating.

14. The implantable prosthesis of claim 1, wherein the implantable prosthesis is formed of a bioresorbable material.

15. The implantable prosthesis of claim 1, wherein the opening is offset from a longitudinal axis of the channel.

16. A method of repairing and/or preventing a parastomal hernia, the method comprising:
placing an implantable prosthesis adjacent to a stoma, the implantable prosthesis comprising a prosthesis body and a channel formed on the prosthesis body having a three-dimensional configuration; and
passing a portion of a bowel through an opening formed in the channel such that the portion of the bowel is lateralized and received in the channel, wherein the opening is formed at a location spaced apart from an end portion of the channel.

17. The method of claim 16, wherein the opening includes a plurality of slits formed in the channel.

18. The method of claim 17, wherein the opening is a cruciate opening.

19. The method of claim 17, wherein the opening includes a first cutout, wherein the plurality of slits extend out from the first cutout, wherein one or more secondary cutouts are formed at an end portion of each slit of the plurality of slits.

20. The method of claim 16, wherein the channel extends inwardly from an outer periphery of the prosthesis body towards an inner region of the prosthesis body.

21. The method of claim 20, wherein the prosthesis body includes a planar portion that extends outwardly from a perimeter of the channel towards the outer periphery.

22. The method of claim 16, wherein the prosthesis body has a width greater than or equal to 10 cm and less than or equal to 50 cm.

23. The method of claim 16, wherein the opening has a width of greater than or equal to 1.5 cm and less than or equal to 5.5 cm, and a height of greater than or equal to 1.5 cm and less than or equal to 5.5 cm.

24. The method of claim 16, wherein the channel has a length greater than or equal to 4 cm and less than or equal to 15 cm and a width greater than or equal to 1 cm and less than or equal to 6 cm.

25. The method of claim 16, wherein the end portion is a first end portion, and further comprising a second end portion of the channel positioned opposite to the first end portion.

26. The method of claim 25, wherein the second end portion is a closed end portion.

27. The method of claim 25, wherein the second end portion is an open end portion.

28. The method of claim 16, wherein the channel includes an adhesion resistant coating.

29. The method of claim 16, wherein the implantable prosthesis is formed of a bioresorbable material.

30. The method of claim 16, wherein the opening is offset from a longitudinal axis of the channel.
